(19) United States
(12) Patent Application Publication
(10) Pub. No.: US 2012/0070468 A1
(43) Pub. Date: Mar. 22, 2012

(54) REMOVAL OF TOXINS FROM GASTROINTESTINAL FLUIDS

(75) Inventors: Robert L. Bedard, McHenry, IL (US); Michael G. Gatter, Elk Grove Village, IL (US)

(73) Assignee: UOP LLC, Des Plaines, IL (US)

(21) Appl. No.: 13/211,012

(22) Filed: Aug. 16, 2011

Related U.S. Application Data
(60) Provisional application No. 61/383,483, filed on Sep. 16, 2010.

(51) Int. Cl. A61K 33/24; A61P 1/00; A61P 39/02; A61K 9/00

(52) U.S. Cl. 424/400; 424/650; 424/617

(57) ABSTRACT

A process for the removal of toxic cations and anions from gastrointestinal fluids is disclosed. A pH-increasing medication is administered prior to or together with a microporous cation exchanger. An additional feature of the invention is the use of a proton form of the microporous cation exchanger. The acidity of the gastrointestinal fluids is decreased to improve the stability of the microporous cation exchangers, which are represented by the empirical formula:

AₚMₓZr₁₋ₓSiₙGe_{y}Oₘ (I)

or

AₚMₓTi₁₋ₓSiₙGe_{y}Oₘ (II)

### CROSS-REFERENCE TO RELATED APPLICATION

This application claims priority from Provisional Application No. 61/383,483 filed Sep. 16, 2010, the contents of which are hereby incorporated by reference.

### BACKGROUND OF THE INVENTION

This invention relates to a process for removing toxins from gastrointestinal fluids. The fluid is contacted with a microporous ion exchange composition to remove toxins such as potassium or ammonium ions. A pH-increasing medication is administered in conjunction with the microporous ion exchange composition to maintain the effectiveness of that composition.

The prior art includes several patents that disclose the use of microporous exchange compositions to remove toxic cations and anions from blood or dialysate, including U.S. Pat. No. 6,579,460, U.S. Pat. No. 6,099,737 and U.S. Pat. No. 6,332,985, incorporated herein in their entirety.

Applicants have developed a process which uses microporous ion exchangers in combination with pH-increasing medications to remove toxins from the body when administered to gastrointestinal fluids. These microporous ion exchangers have an empirical formula on an anhydrous basis of:

AₚMₓZr₁₋ₓSiₙGe_{y}Oₘ (I)

or

AₚMₓTi₁₋ₓSiₙGe_{y}Oₘ (II)

where A is an exchangeable cation selected from the group consisting of potassium ion, sodium ion, rubidium ion, cesium ion, calcium ion, magnesium ion, hydronium ion or mixtures thereof, M is at least one framework metal selected from the group consisting of hafnium (4+), tin (4+), niobium (5+), titanium (4+), cerium (4+), germanium (4+), praseodymium (4+), and terbium (4+), except that M is not titanium in formula (II), “p” has a value from about 1 to about 20, “x” has a value from zero to less than 1, “n” has a value from about 0 to about 12, “y” has a value from 0 to about 12, “m” has a value from about 3 to about 36 and 1≦n+y≦12. The germanium can substitute for the silicon, zirconium/titanium or combinations thereof. Since these compositions are essentially insoluble in bodily fluids (at neutral or basic pH), they can be orally ingested in order to remove toxins in the gastrointestinal system.

### SUMMARY OF THE INVENTION

As stated, this invention relates to a process for removing toxins from gastrointestinal fluids, the process comprising contacting the fluid containing the toxins with a microporous ion exchanger at ion exchange conditions thereby removing the toxins from the fluid. A pH-increasing medication is administered together with the microporous ion exchanger due to the very low pH levels that are found in gastrointestinal fluids that damage or compromise the effectiveness of the microporous ion exchangers.

The microporous ion exchanger is selected from the group consisting of zirconium metallate, titanium metallate and mixtures thereof, the metallates respectively having an empirical formula on an anhydrous basis of:

AₚMₓZr₁₋ₓSiₙGe_{y}Oₘ (I)

and

AₚMₓTi₁₋ₓSiₙGe_{y}Oₘ (II)

where A is an exchangeable cation selected from the group consisting of potassium ion, sodium ion, calcium ion, magnesium ion and mixtures thereof, M is at least one framework metal selected from the group consisting of hafnium (4+), tin (4+), niobium (5+), titanium (4+), cerium (4+), germanium (4+), praseodymium (4+), and terbium (4+), except that M is not titanium in formula (II), “p” has a value from about 1 to about 20, “x” has a value from zero to less than 1, “n” has a value from about 0 to about 12, “y” has a value from 0 to about 12, “m” has a value from about 3 to about 36 and 1≦n+y≦12.

### DETAILED DESCRIPTION OF THE INVENTION

As stated, applicants have developed a new process for removing various toxins from gastrointestinal fluids. One essential element of the process is a microporous ion exchanger which has a large capacity and strong affinity, i.e., selectivity for at least ammonia. These microporous compositions are identified as zirconium metallate and titanium metallate compositions. They are further identified by their empirical formulas (on an anhydrous basis) which respectively are:

AₚMₓZr₁₋ₓSiₙGe_{y}Oₘ (I)

or

AₚMₓTi₁₋ₓSiₙGe_{y}Oₘ (II)

In the case of formula I, the composition has a microporous framework structure composed of ZrO₃ octahedral units and at least one of SiO₂ tetrahedral units and GeO₂ tetrahedral units. In the case of formula II, the microporous framework structure is composed of TiO₃ octahedral units and at least one of SiO₂ tetrahedral units and GeO₂ tetrahedral units.

In both formulas I and II, A is an exchangeable cation selected from the group consisting of potassium ion, sodium ion, rubidium ion, cesium ion, calcium ion, magnesium ion, hydronium ion or mixtures thereof, M is at least one framework metal selected from the group consisting of hafnium (4+), tin (4+), niobium (5+), titanium (4+), cerium (4+), germanium (4+), praseodymium (4+), and terbium (4+), “p” has a value from about 1 to about 20, “x” has a value from zero to less than 1, “n” has a value from about 0 to about 12, “y” has a value from 0 to about 12, “m” has a value from about 3 to about 36 and the sum of n+y has a value from about 1 to about 12. That is 1≦n+y≦12. In equation (II) M is, of course, not titanium. The M metals which can be inserted into the framework in place of zirconium will be present as MO₃ octahedral units and thus it is a requirement that they are capable of being octahedrally coordinated. The germanium can be inserted into the framework in place of silicon and will be present as MO₂ tetrahedral units. Additionally, germanium can be inserted into the framework as a MO₃ octahedral unit replacing some of the zirconium in formula (I) or some of the titanium in formula (II). That is, germanium can replace some or all of the silicon, some of the zirconium in formula (I), some of the titanium in formula (II) or both silicon and zirconium or both silicon and titanium.

The zirconium metallates are prepared by a hydrothermal crystallization of a reaction mixture prepared by combining a reactive source of zirconium, silicon and/or germanium, optionally one or more M metal, at least one alkali metal and water. The alkali metal acts as a templating agent. Any zirconium compound, which can be hydrolyzed to zirconium oxide or zirconium hydroxide, can be used. Specific examples of these compounds include zirconium alkoxide, e.g., zirconium n-propoxide, zirconium hydroxide, zirconium acetate, zirconium oxychloride, zirconium chloride, zirconium phosphate and zirconium oxynitrate. The sources of silica include colloidal silica, fumed silica and sodium silicate. The sources of germanium include germanium oxide, germanium alkoxides and germanium tetrachloride. Alkali sources include potassium hydroxide, sodium hydroxide, rubidium hydroxide, cesium hydroxide, sodium carbonate, potassium carbonate, rubidium carbonate, cesium carbonate, sodium halide, potassium halide, rubidium halide, cesium halide, sodium ethylenediamine tetraacetic acid (EDTA), potassium EDTA, rubidium EDTA, and cesium EDTA. The M metals sources include the M metal oxides, alkoxides, halide salts, acetate salts, nitrate salts and sulfate salts. Specific examples of the M metal sources include, but are not limited to titanium alkoxides, titanium tetrachloride, titanium trichloride, titanium dioxide, tin tetrachloride, tin isopropoxide, niobium isopropoxide, hydrous niobium oxide, hafnium isopropoxide, hafnium chloride, hafnium oxychloride, cerium chloride, cerium oxide and cerium sulfate.

The titanium metallates are prepared in an analogous manner to the zirconium metallates. Thus, the sources of silicon, germanium, M metal and alkali metal are as enumerated above. The titanium source is also as enumerated above, namely titanium alkoxides, titanium tetrachloride, titanium trichloride and titanium dioxide. A preferred titanium source is titanium alkoxides with specific examples being titanium isopropoxide, titanium ethoxide and titanium butoxide.

Generally, the hydrothermal process used to prepare the zirconium metallate or titanium metallate ion exchange compositions of this invention involves forming a reaction mixture which in terms of molar ratios of the oxides is expressed by the formulae:

a A₂O:b MO_{q/2}:1−b ZrO₂:c SiO₂:d GeO₂:e H₂O (III)

and

a A₂O:b MO_{q/2}:1−b TiO₂:c SiO₂:d GeO₂:e H₂O (IV)

where “a” has a value from about 0.25 to about 40, “b” has a value from about 0 to about 1, “q” is the valence of M, “c” has a value from about 0.5 to about 30, “d” has a value from about 0 to about 30 and “e” has a value of 10 to about 3000. The reaction mixture is prepared by mixing the desired sources of zirconium, silicon and optionally germanium, alkali metal and optional M metal in any order to give the desired mixture. It is also necessary that the mixture have a basic pH and preferably a pH of at least 8. The basicity of the mixture is controlled by adding excess alkali hydroxide and/or basic compounds of the other constituents of the mixture. Having formed the reaction mixture it is next reacted at a temperature of about 100° C. to about 250° C. for a period of about 1 to about 30 days in a sealed reaction vessel under autogenous pressure. After the allotted time, the mixture is filtered to isolate the solid product which is washed with deionized water and dried in air.

As stated the microporous compositions of this invention have a framework structure of octahedral ZrO₃ units, at least one of tetrahedral SiO₂ units and tetrahedral GeO₂ units and optionally octahedral MO₃ units. This framework results in a microporous structure having an intracrystalline pore system with uniform pore diameters, i.e., the pore sizes are crystallographically regular. The diameter of the pores can vary considerably from about 3 Å and larger.

As synthesized, the microporous compositions of this invention will contain some of the alkali metal templating agent in the pores. These metals are described as exchangeable cations, meaning that they can be exchanged with other (secondary) A′ cations. Generally, the A exchangeable cations can be exchanged with A′ cations selected from other alkali metal cations (K⁺, Na⁺, Rb⁺, Cs⁺), alkaline earth cations (Mg²⁺, Ca²⁺, Sr²⁺, Ba²⁺), hydronium ion or mixtures thereof. It is understood that the A′ cation is different from the A cation. The methods used to exchange one cation for another are well known in the art and involve contacting the microporous compositions with a solution containing the desired cation (at molar excess) at exchange conditions. Exchange conditions include a temperature of about 25° C. to about 100° C. and a time of about 20 minutes to about 2 hours. The particular cation (or mixture thereof) which is present in the final product will depend on the particular use and the specific composition being used. One specific composition is an ion exchanger where the A′ cation is a mixture of Na⁺, Ca⁺² and H⁺ ions.

In a preferred embodiment of the invention, the ion exchanger is in a sodium form which is much more effective than other forms of the ion exchanger.

It is also within the scope of the invention that these microporous ion exchange compositions can be used in powder form or can be formed into various shapes by means well known in the art. Examples of these various shapes include pills, extrudates, spheres, pellets and irregularly shaped particles.

As stated, these compositions have particular utility in adsorbing various toxins from fluids selected from gastrointestinal fluids. These compositions have utility in treatment of any mammalian body including but not limited to humans, cows, pigs, sheep, monkeys, gorillas, horses, dogs, etc. The instant process is particularly suited for removing toxins from a human body.

The zirconium metallates and titanium metallates can also be formed into pills or other shapes which can be ingested orally and pickup toxins in the gastrointestinal fluid as the ion exchanger passes through the intestines and is finally excreted. It has been found important to increase the pH level of the gastrointestinal fluids in order for the ion exchangers to retain their efficacy in removal of toxins. Among the pH increasing medications that may be used are antacids such as sodium bicarbonate, potassium carbonate, aluminum hydroxide, magnesium hydroxide, calcium carbonate, bismuth salicylate and mixtures thereof; histamine H₂ receptor blockers such as cimetidine, ranitidine, famotidine and nizatidine; and proton pump inhibitors such as omeprazole, lansoprazole, dexlansoprazole, esomeprazole, pantoprazole and rabeprazole.

As has also been stated, although the instant compositions are synthesized with a variety of exchangeable cations (“A”), it is preferred to exchange the cation with secondary cations (A′) which are more compatible with blood or do not adversely affect the blood. For this reason, preferred cations are sodium, calcium, hydronium and magnesium. Preferred compositions are those containing sodium and calcium or sodium, calcium and hydronium ions. The relative amount of sodium and calcium can vary considerably and depends on the microporous composition and the concentration of these ions in the blood.

In order to more fully illustrate the invention, the following examples are set forth. It is to be understood that the examples are only by way of illustration and are not intended as an undue limitation on the broad scope of the invention as set forth in the appended claims.

### EXAMPLE 1

A solution was prepared by mixing 2058 g of colloidal silica (DuPont Corp. identified as Ludox® AS-40), 2210 g of KOH in 7655 g H₂O. After several minutes of vigorous stirring 1471 g of a zirconium acetate solution (22.1 wt-% ZrO₂) were added. This mixture was stirred for an additional 3 minutes and the resulting gel was transferred to a stainless steel reactor and hydrothermally reacted for 36 hours at 200° C. The reactor was cooled to room temperature and the mixture was vacuum filtered to isolate solids which were washed with deionized water and dried in air.

The solid reaction product was analyzed and found to contain 21.2 wt-% Si, 21.5 wt-% Zr, K 20.9 wt-% K, LOI 12.8 wt-%, which gave a formula of K_{2.3}ZrSi_{3.2}O_{9.5}*3.7H₂O. This product was identified as sample A.

### EXAMPLE 2

A solution was prepared by mixing 121.5 g of colloidal silica (DuPont Corp. identified as Ludox® AS-40), 83.7 g of NaOH in 1051 g H₂O. After several minutes of vigorous stirring 66.9 g zirconium acetate solution (22.1 wt-% ZrO₂) was added. This was stirred for an additional 3 minutes and the resulting gel was transferred to a stainless steel reactor and hydrothermally reacted with stirring for 72 hours at 200° C. The reactor was cooled to room temperature and the mixture was vacuum filtered to isolate solids which were washed with deionized water and dried in air.

The solid reaction product was analyzed and found to contain 22.7 wt-% Si, 24.8 wt-% Zr, 12.8 wt-% Na, LOI 13.7 wt-%, which gives a formula Na_{2.0}ZrSi_{3.0}O_{9.0}*3.5H₂O. This product was identified as sample B.

### EXAMPLE 3

A solution (60.08 g) of colloidal silica (DuPont Corp. identified as Ludox® AS-40) was slowly added over a period of 15 minutes to a stirring solution of 64.52 g of KOH dissolved in 224 g deionized H₂O. This was followed by the addition of 45.61 g zirconium acetate (Aldrich 15-16 wt-% Zr, in dilute acetic acid). When this addition was complete, 4.75 g hydrous Nb₂O₅ (30 wt-% LOI) was added and stirred for an additional 5 minutes. The resulting gel was transferred to a stirred autoclave reactor and hydrothermally treated for 1 day at 200° C. After this time, the reactor was cooled to room temperature, the mixture was vacuum filtered, the solid washed with deionized water and dried in air.

The solid reaction product was analyzed and found to contain 20.3 wt-% Si, 15.6 wt-% Zr, 20.2 wt-% K, 6.60 wt-% Nb, LOI 9.32 wt-%, which give a formula of K_{2.14}Zr_{0.71}Nb_{0.29}Si₃O_{9.2}•2.32 H₂O. Scanning Electron Microscopy (SEM) of a portion of the sample, including EDAX of a crystal, indicated the presence of niobium, zirconium, and silicon framework elements. This product was identified as sample C.

### EXAMPLE 4

To a solution prepared by mixing 141.9 g of NaOH pellets in 774.5 g of water, there were added 303.8 g of sodium silicate with stirring. To this mixture there were added dropwise, 179.9 g of zirconium acetate (15% Zr in a 10% acetic acid solution). After thorough blending, the mixture was transferred to a Hastalloy™ reactor and heated to 200° C. under autogenous pressure with stirring for 72 hours. At the end of the reaction time, the mixture was cooled to room temperature, filtered and the solid product was washed with a 0.001M NaOH solution and then dried at 100° C. for 16 hours. Analysis by x-ray powder diffraction showed that the product was pure UZSi-11.

### EXAMPLE 5

To a container there was added a solution of 37.6 g NaOH pellets dissolved in 848.5 g water and to this solution there were added 322.8 g of sodium silicate with mixing. To this mixture there were added dropwise 191.2 g of zirconium acetate (15% Zr in 10% acetic acid). After thorough blending, the mixture was transferred to a Hastalloy™ reactor and the reactor was heated to 200° C. under autogenous conditions with stirring for 72 hours. Upon cooling, the product was filtered, washed with 0.001 M NaOH solution and then dried at 100° C. for 16 hours. X-ray powder diffraction analysis showed the product to be UZSi-9.

The most straightforward way to make H-UZSi-9 from Na-UZSi-9 is to treat the Na-form with aqueous HCl solution. However, Na-UZSi-9 is susceptible to decomposition in strong acids. It was found that Na-UZSi-9 is unstable in HCl solution with concentrations greater than 0.2 M at room temperature as evidenced by partial or complete structure collapse after overnight exposure. It has been observed that while UZSi-9 has borderline stability in 0.2 M HCl at room temperature, rapid crystallinity loss occurs after about 20 minutes at 37° C. (simulated gastric fluid temperature). However, the Na-UZSi-9 survives in room temperature solutions of 0.1 M HCl and the Na level is decreased from 13 to 2% after overnight treatment. The H-form of UZSi-9 can be made by subjecting Na-UZSi-9 to three batch-wise ion exchanges with 0.1 M HCl using the following procedure:

First add 2.0 g (non-volatile-free basis) of Na-UZSi-9 to 200 mL of 0.1 M HCl. Gently stir the slurry with a stir bar at room temperature for 30 minutes and then decant off the HCl solution. Repeat this procedure two more times with fresh 0.1 M HCl and after the third exchange, dry the powder at 100° C. The resulting H-UZSi-9 has 0.053% Na. Alternatively, the H-UZSi-9 can be made by ammonium exchange of Na-UZSi-9 followed by calcination, although the crystallinity of the final product made this way is significantly lower than the HCl exchanged product. Three successive ammonium exchanges using 1 g of NH₄NO₃ per gram of Na-UZSi-9 in 10 g of H₂O for 3 hours at 85° C. yields NH4-UZSi-9 with 0.05% Na. Calcination at 350° C. for two hours would form the H-form of UZSi-9. This alternative is not preferred due to the low heat stability of both the NH4+ and H+ forms of UZSi-9.

1. A process for removing toxins from gastrointestinal fluid, the process comprising contacting the fluid with a shaped ion exchange composite at ion exchange conditions together with a pH-increasing medication, thereby providing a purified fluid, the composite comprising a mixture of a microporous cation exchange composition and an anion exchange composition, where the cation exchange composition is selected from the group consisting of zirconium metallate, titanium metallate and mixtures thereof, the metallates respectively having an empirical formula on an anhydrous basis of:
AₚMₓZr₁₋ₓSiₙGe_{y}Oₘ (I)
or
AₚMₓTi₁₋ₓSiₙGe_{y}Oₘ (II)
where A is an exchangeable cation selected from the group consisting of potassium ion, sodium ion, calcium ion, magnesium and mixtures thereof, M is at least one framework metal selected from the group consisting of hafnium (4+), tin (4+), niobium (5+), titanium (4+), cerium (4+), germanium (4+), praseodymium (4+), and terbium (4+), except that M is not titanium in formula (II), “p” has a value from about 1 to about 20, “x” has a value from zero to less than 1, “n” has a value from 0 to about 12, “y” has a value from 0 to about 12, “m” has a value from about 3 to about 36 and 1≦n+y≦12, and the anion exchange composition is selected from the group consisting of hydrous zirconium oxide, zirconia, alumina, titania, hydrous titanium oxide, layered double hydroxides, single phase metal oxide solid solutions, magnesium hydroxide, calcium hydroxide, silica, amorphous mixed metal oxides, basic clays and mixtures thereof.

2. The process of claim 1 wherein the pH-increasing medication is selected from the group consisting of antacids, alginic acid, histamine H₂ receptor blockers, proton pump inhibitors and mixtures thereof.

3. The process of claim 2 wherein the pH-increasing medication comprises an antacid selected from the group consisting of sodium bicarbonate, potassium carbonate, aluminum hydroxide, magnesium hydroxide, calcium carbonate, bismuth subsalicylate, and mixtures thereof.

4. The process of claim 1 wherein said pH-increasing medication increases the pH of said gastrointestinal fluids by from about 3 to 4 pH units.

5. The process of claim 1 wherein said pH-increasing medication increases the pH of said gastrointestinal fluids to about 2 to 6.

6. The process of claim 2 wherein the pH-increasing medication comprises at least one histamine H₂ receptor blocker selected from the group consisting of cimetidine, ranitidine, famotidine, and nizatidine.

7. The process of claim 2 wherein the pH-increasing medication comprises at least one proton pump inhibitor selected from the group consisting of omeprazole, lansoprazole, dexlansoprazole, esomeprazole, pantoprazole and rabeprazole.

8. The process of claim 1 where the ion exchange composite is in a shape selected from extrudates, pills, pellets, spheres and irregularly shaped particles.

9. The process of claim 1 where the composite is further characterized in that it contains a binder selected from the group consisting of hydrous zirconium oxide, zirconia, zirconium phosphate, alumina, aluminum phosphate, titania, titanium phosphate, hydrous titanium oxide, layered double hydroxides, magnesium hydroxide, calcium hydroxide, silica, basic clays and mixtures thereof.

10. The process of claim 1 wherein the toxin comprises potassium ions.

11. The process of claim 1 where the toxin comprises ammonium ions.

12. The process of claim 1 where the toxins are ammonium and phosphate ions.

13. The process of claim 1 where M is tin (4+).

14. The process of claim 1 where M is titanium (4+).

15. The process of claim 1 where M is niobium (5+).

16. The process of claim 1 where n=0.

17. The process of claim 1 further characterized in that the A cation is exchanged for a different secondary cation, A′, selected from the group consisting of alkali metals, alkaline earth metal, hydronium ions and mixtures thereof.

18. The process of claim 17 where A′ comprises sodium ions.

19. The process of claim 17 where A′ is a mixtures of sodium and calcium ions.

20. The process of claim 17 where A′ is a mixture of sodium, calcium and hydronium ions.

21. The process of claim 1 where the cation exchange composition has the structure of UZSi-9, UZSi-11 or UZSi-1.

22. A process for removing toxins from a mammalian body comprising enterally administering a microporous ion exchanger and a pH-increasing medication wherein the microporous ion exchanger is selected from the group consisting of zirconium metallate, titanium metallate and mixtures thereof, the metallates respectively having an empirical formula on an anhydrous basis of:
AₚMₓZr₁₋ₓSiₙGe_{y}Oₘ (I)
or
AₚMₓTi₁₋ₓSiₙGe_{y}Oₘ (II)
where A is an exchangeable cation selected from the group consisting of potassium ion, sodium ion, calcium ion, magnesium and mixtures thereof, M is at least one framework metal selected from the group consisting of hafnium (4+), tin (4+), niobium (5+), titanium (4+), cerium (4+), germanium (4+), praseodymium (4+), and terbium (4+), except that M is not titanium in formula (II), “p” has a value from about 1 to about 20, “x” has a value from zero to less than 1, “n” has a value from 0 to about 12, “y” has a value from 0 to about 12, “m” has a value from about 3 to about 36 and 1≦n+y≦12, and the anion exchange composition is selected from the group consisting of hydrous zirconium oxide, zirconia, alumina, titania, hydrous titanium oxide, layered double hydroxides, single phase metal oxide solid solutions, magnesium hydroxide, calcium hydroxide, silica, amorphous mixed metal oxides, basic clays and mixtures thereof and the pH-increasing medication is selected from the group consisting of antacids, alginic acid, histamine H₂ receptor blockers, proton pump inhibitors and mixtures thereof.

23. A process for removing toxins from gastrointestinal fluid, the process comprising first administering a pH-increasing medication to a patient to increase the pH of the gastrointestinal fluid and then contacting the gastrointestinal fluid with a shaped ion exchange composite at ion exchange conditions, thereby providing a purified fluid, the composite comprising a mixture of a microporous cation exchange composition and an anion exchange composition, where the cation exchange composition is selected from the group consisting of zirconium metallate, titanium metallate and mixtures thereof, the metallates respectively having an empirical formula on an anhydrous basis of:
AₚMₓZr₁₋ₓSiₙGe_{y}Oₘ (I)
or
AₚMₓTi₁₋ₓSiₙGe_{y}Oₘ (II)
where A is an exchangeable cation selected from the group consisting of potassium ion, sodium ion, calcium ion, magnesium and mixtures thereof, M is at least one framework metal selected from the group consisting of hafnium (4+), tin (4+), niobium (5+), titanium (4+), cerium (4+), germanium (4+), praseodymium (4+), and terbium (4+), except that M is not titanium in formula (II), “p” has a value from about 1 to about 20, “x” has a value from zero to less than 1, “n” has a value from 0 to about 12, “y” has a value from 0 to about 12, “m” has a value from about 3 to about 36 and 1≦n+y≦12, and the anion exchange composition is selected from the group consisting of hydrous zirconium oxide, zirconia, alumina, titania, hydrous titanium oxide, layered double hydroxides, single phase metal oxide solid solutions, magnesium hydroxide, calcium hydroxide, silica, amorphous mixed metal oxides, basic clays and mixtures thereof.
